(19) United States
(12) Patent Application Publication
(10) Pub. No.: US 2019/0131003 A1
(43) Pub. Date: May 2, 2019

(54) PROCESS FOR CARRYING OUT A CHANGE IN AN ASSIGNMENT OF ASSIGNED PATIENTS IN A CARE SYSTEM AS WELL AS CARE SYSTEM

(71) Applicant: Drägerwerk AG & Co. KGaA, Lübeck (DE)

(72) Inventors: Tobias KLOTZ, Lübeck (DE); Maximilian MERKEL, Hamburg (DE)

(21) Appl. No.: 16/177,911

(22) Filed: Nov. 1, 2018

(30) Foreign Application Priority Data
Nov. 2, 2017 (DE) 10 2017 010 149.3

(51) Int. Cl. G16H 10/60; H04M 1/725; G16H 50/20; G16H 80/00

(52) U.S. Cl. CPC G16H 10/60; H04M 1/7258; G16H 15/00; G16H 80/00; G16H 50/20

(57) ABSTRACT

A process caries out a change (50) of an assignment of patients (1, 2) in a care system (100) on mobile terminals (20, 30). The care system (100) includes at least one central computer (10) for monitoring and documenting the assignment of the patients (1, 2) as well as at least one first mobile terminal (20) and a second mobile terminal (30). Patients (1, 2) can each be assigned by the central computer (10) to the mobile terminals (20, 30).

### CROSS REFERENCE TO RELATED APPLICATIONS

This application claims the benefit of priority under 35 U.S.C. § 119 of German Application 10 2017 010 149.3, filed Nov. 2, 2017, the entire contents of which are incorporated herein by reference.

### TECHNICAL FIELD

The present invention pertains to a process for carrying out a change of an assignment of patients in a care system on mobile terminals, the care system comprising at least one central computer for monitoring and documenting the assignment of the patients as well as at least one first mobile terminal and a second mobile terminal, wherein patients can each be assigned by the central computer to the mobile terminals. In addition, the present invention pertains to a care system for monitoring patient s, comprising at least one central computer for monitoring and documenting the assignment of the patients, as well as at least one first mobile terminal and a second mobile terminal, wherein patients are each assigned by the central computer to the mobile terminals.

### BACKGROUND

It is known, in principle, in modern medical engineering that patients are monitored by apparatuses. Such a monitoring may pertain to data characterizing the status of the patient, for example, a blood pressure, a heart rate and/or an oxygen saturation in the blood of the patient. The monitoring may comprise, in particular, the triggering of alarms when the patient data being monitored exceed or drop below limit values. This is carried out essentially usually by the monitoring apparatuses themselves, which are arranged in the vicinity of the patient's bed.

Care systems, which are configured for distributing and transmitting the alarms, are known in the state of the art in order to be able to provide location-independent information about such a present alarm, for example, for care staff. Such care systems usually have a central computer and at least two mobile terminals, wherein the individual mobile terminals are usually assigned to a user, for example, to a doctor or to a nurse or to a health care staff member, who are in turn often responsible for a defined group of patients. In other words, the patients are thus usually also assigned to one of the mobile terminals. The mobile terminals may especially be carried by the user. If an alarm is triggered in case of one of the patients, the corresponding mobile terminal, to which the patient is assigned, is informed by the central computer and the corresponding alarm is additionally outputted by the mobile terminal. A user of the mobile terminal, who is responsible, for example, for the care of the corresponding patient, can thus also be informed about the present alarms via the mobile terminal, especially when he is not directly present in the vicinity of the patient.

To change an assignment of the patients on the individual mobile terminals, for example, at the end of a shift of the corresponding user of the mobile terminal, it is known according to the state of the art, on the one hand, to exchange the corresponding terminals between users. This may especially entail, for example, the drawback that no documentation is carried out during this transfer, and thus in retrospect, it is not comprehensible which user is or was responsible for the respective patient at what time. This direct personal transfer of mobile terminals may also be problematic with regard to a possible passing on of bacteria. Another known possibility according to the state of the art is to change the assignment of the patients on the individual mobile terminals directly by an input at the central computer. However, this can usually only be carried out by specially trained persons or by authorized persons, so that such a transfer or a change of the assignment of the patients on the individual mobile terminals is time-consuming and especially labor-intensive.

### SUMMARY OF THE INVENTION

Based on this state of the art, a basic object of the present invention is to at least partly eliminate these drawbacks of processes for carrying out a change of an assignment of patients in a care system as well as in care systems. Therefore, the object of the present invention is to provide a process for carrying out a change of an assignment of patients in a care system as well as a care system, by means of which such a change of the assignment can be simplified, wherein especially the change can be carried out more rapidly and with a lower effort and in a less time-consuming manner.

The above object is accomplished by a process for carrying out a change of assignment of patients in a care system on mobile terminals having features according to the invention. Further, the object is accomplished by a care system having the features according to the invention. Further features and details of the present invention appear from the description and the drawings. Features and details that are described in connection with the process according to the present invention also apply, of course, in connection with the care system according to the present invention and vice versa, so that reference is and can always mutually be made concerning the different aspects of the present invention.

According to a first aspect of the present invention, the object is accomplished by a process for carrying out a change of an assignment of patients in a care system on mobile terminals, the care system comprising at least one central computer for monitoring and documenting the assignment of the patients as well as at least one first mobile terminal and a second mobile terminal, wherein patients can each be assigned by the central computer to the mobile terminals. A process according to the present invention is characterized by the following steps:
a) generation of a transfer request for a change of the assignment of at least one assigned patient by the first mobile terminal;
b) transmission of the transfer request from the first mobile terminal to the second mobile terminal;
c) transmission of the transfer request to the central computer;
d) carrying out of the change of the assignment of the at least one assigned patient by the central computer; and
e) transmission of the change of the assignment of the at least one assigned patient to the first mobile terminal and to the second mobile terminal.

A process according to the present invention is configured such that a change of an assignment of patients in a care system can be carried out in a simplified manner on mobile terminals. Provisions are made in the process according to the present invention in particular and in a manner essential to the present invention for the transfer request to be initiated directly from a mobile terminal and transmitted to a second mobile terminal. The actual change of the assignment is, furthermore, carried out in the central computer, but an input of the transfer request for this directly at the central computer can be omitted. All safety aspects, which are necessary in case of such a change of the assignment of patients on the mobile terminals and are provided by the central computer, can thus also be ensured by a process according to the present invention. The individual steps of a process according to the present invention will be described in more detail below.

A transfer request is generated on a first mobile terminal in a first step a). The generation may be initiated, for example, by a user of the first mobile terminal, for example, by means of an input via a corresponding input device of the first mobile terminal. The transfer request may especially contain information about which patient it concerns, which user of the first mobile terminal triggered the transfer request, in what manner the assignment shall be changed and/or at which user or at which second mobile terminal the change shall be carried out.

This transfer request generated in step a) is now transmitted from the first mobile terminal to the second mobile terminal in the next step b). The mobile terminals especially have the devices necessary for this in order to carry out such a transmission. After carrying out step b) of a process according to the present invention, information is thus present in the second mobile terminal about the fact that a transfer of an assignment of at least one assigned patient, which was triggered by the first mobile terminal, shall be carried out and about the nature of the change.

Step c), which follows step b), contains a transmission of the transfer request to the central computer. This transmission may be carried out from one of the two mobile terminals or from both mobile terminals. After carrying out step c) of a process according to the present invention, information is thus present in the central computer about the fact that a change of an assignment of patients is desired and how this is configured, from which mobile terminal this desire originates and which mobile terminal will additionally be affected by this change. Further information, especially about which patient or which assignment it concerns, may be present as information in the central computer after step c) as well.

The actual change of the assignment of the at least one assigned patient is now carried out by the central computer in the next step d). In other words, the assignment of the at least one assigned patient is removed from one of the mobile terminals and allocated to the other mobile terminal. Provisions may be made for the assignment of the at least one assigned patient to be transferred from the first mobile terminal to the second mobile terminal or vice versa in this step. A partial change of the assignment may also be carried out in this step, so that, for example, only parts of the assignment of a patient are transferred from one mobile terminal to the other mobile terminal.

As the last step of a process according to the present invention, the carrying out of the change of the assignment of the at least one assigned patient is transmitted back to the mobile terminals in step e). This transmission is carried out both to the first mobile terminal and the second mobile terminal in order to install the reassignment of the at least one assigned patient on the corresponding mobile terminal, on the one hand, and in order to remove the old, especially changed assignment on the corresponding mobile terminal, on the other hand. The change of an assignment of patients in a care system is carried out and completed on the mobile terminals after carrying out step e) of a process according to the present invention.

In summary, provisions may thus be made by a process according to the present invention for a change of an assignment of patients in a care system to be able to be carried out in a simplified manner. In particular, the change of the assignment can be initiated from the individual mobile terminals and especially be carried out without involvement of other, especially authorized persons. A simplification and especially a reduction of effort and amount of time needed in the carrying out of the change of an assignment of patients in a care system on mobile terminals can be provided in this manner.

Provisions may especially preferably be made in a process according to the present invention for one of the following transmission modes to be used in steps b), c) and/or e):
Near Field Communication (NFC);
one-dimensional barcode;
Quick Response (QR) code;
Wireless Local Area Network (WLAN);
wireless device;
mobile cell phone.

This list is especially not exhaustive, so that other transmission modes may also be used in case they are technically useful and possible. As already described above, especially the mobile terminals and, in addition, the central computer also have corresponding devices, for example, cameras, displays and/or antennas, to be able to carry out the individual transmission modes. WLAN, wireless device and/or mobile cell phone are preferably used as transmission modes in steps c) and e), in which data are transferred between the mobile terminals and the central computer. Transmission modes, which require the presence of both mobile terminals, for example, NFC or a display and reading of a barcode or QR code, may also be used in step b). Overall, different and preferably suitably selected transmission modes may be used to be able to carry out the respective transmission in steps b) and c) and/or e) in a simple, rapid and data-secure manner.

In addition, provisions may be made in the process according to the present invention for a checking of the admissibility of the requested change of the assignment of the at least one assigned patient to be carried out by the central computer in step d). It can be ensured in this manner that the requested change is also admissible. Admissible in the sense of the present invention may especially mean whether the first mobile terminal may release an assignment of the respective patient and the second mobile terminal may accept an assignment of the respective patient. A guarantee of a patient's safety, at any time, may be provided in this manner. Additional criteria, for example, a user verification, may also be checked in step d).

A process according to the present invention may preferably be perfected such that the checking carried out in step d) pertains to at least one of the following points:
Location of the first mobile terminal;
location of the second mobile terminal;
patient data of the at least one assigned patient;
user data of a user of the first mobile terminal;
user data of a user of the second mobile terminal.

This list is especially not exhaustive, so that additional points may also be included in the checking carried out. By checking the locations of the first and/or second mobile terminal, it can especially be ensured that during the change of the assignment of the at least one assigned patient, this patient is not assigned to a mobile terminal that is located, for example, in a completely different area. In particular, an assignment of the respective patient to a user of a mobile terminal, who is located far away and thus must, for example, first overcome a great distance to the patient if an alarm has been triggered, can thereby be avoided. Patient data, for example, a health status or the course of disease of the patient, may, for example, especially be used in connection with the user data of the user of the terminals in order to correlate whether the users, especially the user of the receiving mobile terminal, are/is qualified for a takeover. A qualification of the user for a takeover of the assignment of the at least one assigned patient may also be checked due to the inclusion of user data of the user of the terminals. In addition, the user data may also contain an identification or verification of the respective user of the mobile terminal. A work area, a work load, a training and/or responsibilities may also represent parts of the user data of the users of the mobile terminals. A patient's safety can thus especially be increased by checking these user data, since only new patients can be assigned to the mobile terminals of those users who are also qualified for this assignment.

Provisions may especially preferably be made in a process according to the present invention for a documentation of the change carried out to be carried out by the central computer in step d). Such a documentation may represent, for example, a storage and/or printing out of the change carried out. An assignment of the patient to the mobile terminals and thus to the corresponding users of the mobile terminals can thus also be traced back at any time for each time in retrospect. Care of the patient and responsibilities can thus also be traced back and especially be checked at a later time.

Further, provisions may be made in a process according to the present invention for a change of an assignment of two or more assigned patients, and especially of all patients assigned to the first mobile terminal, to be requested in step a). In this manner, it can be made possible that a separate transfer request does not have to be generated for each individual patient. For example, when the user of the first mobile terminal wants to transfer all patients assigned to him to another mobile terminal at the end of a shift, this can be carried out in a rapid and simple manner in this preferred embodiment of a process according to the present invention.

In addition, transfer of only one patient or of only some of the patients assigned to a mobile terminal and hence to a user is also conceivable, for example, in order to split up the patients from one mobile terminal to a plurality of mobile terminals and to assign them to the users of the mobile terminals.

A process according to the present invention may also be configured such that the transmission to the central computer in step c) is carried out by the first mobile terminal and/or by the second mobile terminal. All three different transmissions are possible and conceivable, as a result of which step c) of a process according to the present invention can be carried out in an especially simple manner. A transmission to the central computer by the second mobile terminal can especially be carried out after receipt of the transfer request by the second mobile terminal. A transmission to the central computer by the first mobile terminal may especially preferably be carried out after an acknowledgment from the second mobile terminal to the first mobile terminal about an acceptance of the transfer request. A redundancy of this transmission may especially be provided by a transmission to the central computer by both mobile terminals. A transfer or a change of the assignment may preferably be carried out in this case only if both transmissions have been received by the central computer, as a result of which the safety during the change of the assignment and thus in the entire care system can be increased in turn.

Further, a process according to the present invention may be characterized in that a consent for the requested change of the assignment of the at least one assigned patient is checked by the second mobile terminal before step c) and/or before step d). Such a consent may be generated, for example, by an input of a user at the second mobile terminal. It can thereby especially be ensured that the user of the second mobile terminal is also informed about the change of the assignment. A carrying out of a change of the assignment of patients on the mobile terminals without information or consent of all users of the participating mobile terminals can be reliably avoided in this manner.

A process according to the present invention may also be configured such that the change of the assignment of the at least one assigned patient is carried out in a manner limited in time and/or in place in step d). Thus, for example, the change of the assignment may preferably be reversed again after a certain time, for example, when the user of the first mobile terminal has requested or needs a change of the assignment only for a brief time, for example, for a work break. Carrying out the change in a locally limited manner may, for example, be advantageous because the change is withdrawn as soon as the first mobile terminal again returns, for example, to an area around the patient. This may also be the case, for example, after a work break of a user of the first mobile terminal or else also, for example, after return after a patient transfer. In both cases, it is especially possible to avoid that a new transfer desire has to be expressed for a changing back of the assignment. The changing back of the assignment may be carried out automatically in this preferred embodiment of a process according to the present invention.

Further, provisions may be made in the process according to the present invention for patient data of the at least one reassigned patient to be transmitted from the central computer to the second mobile terminal in step e). Such patient data may especially be alarms, a health status and/or a course of disease of the respective patient. A keeping available of the patient data on the currently assigned mobile terminal can especially be ensured in this manner.

As an alternative or in addition, provisions may further be made in the process according to the present invention for patient data of the at least one reassigned patient to be transmitted from the first mobile terminal to the second mobile terminal in step e). A keeping available of patient data on the currently assigned mobile terminal may also be ensured in this case.

Should a change of the assignment of the patient on the mobile terminals comprise a change of the assignment from the second mobile terminal to the first mobile terminal, the described transmissions of patient data may, of course, also be carried out correspondingly from the central computer to the first mobile terminal or from the second mobile terminal to the first mobile terminal.

A process according to the present invention may especially preferably be configured such that the transmission in step b) and/or c) and e) is carried out in an encrypted manner (e.g., with the computer and the mobile terminal following an encryption scheme in which communications are encrypted using one or more encryption key and the recipient can decrypt the communication message with the key provided by the originator to the recipient). An access to the transmitted data by unauthorized third parties can be reliably avoided in this way. An increase in the safety of the entire care system and especially a protection of the data of the patient can be made possible in this way.

A process according to the present invention may also be configured such that the assignment of the at least one assigned patient is changed entirely or partially during the change of the assignment of the at least one assigned patient. By means of a complete change, the responsibility for the at least one assigned patient is completely transferred to the new mobile terminal and thus to a new user. A partial assignment of the patient to different mobile terminals is also possible due to a splitting up of the assignment. Thus, for example, alarms, which are assigned as patient data to the patient and, for example, concern different organ systems of the patient, can be assigned to different mobile terminals, the respective users of which have the correspondingly necessary qualifications. A flexibility in the assignment of patients to the individual mobile terminal may be increased as a result.

A process according to the present invention may also be configured such that the assignment of the at least one assigned patient is changed during the change of the assignment of the at least one assigned patient from an assignment to the first mobile terminal to an assignment to the second mobile terminal and/or the assignment of the at least one assigned patient is changed during the change of the assignment of the at least one assigned patient from an assignment to the second mobile terminal to an assignment to the first mobile terminal. In this way, it can especially be made possible that the generation of a transfer request in step a) also comprises the generation of a transmission request. Thus, a request may also be started by the first mobile terminal to take over an assignment of a patient from the second mobile terminal. A flexibility in the carrying out of a process according to the present invention, especially with regard to a change in an assignment of patients to the different mobile terminals, can be increased as a result. The process steps and advantages described above also appear analogously in this embodiment of a process according to the present invention.

According to a second aspect of the present invention, the object is accomplished by a care system for monitoring patients, comprising at least one central computer for monitoring and documenting the assignment of the patients, as well as at least one first mobile terminal and a second mobile terminal, wherein patients are each assigned by the central computer to the mobile terminals. A care system according to the present invention is characterized in that the care system is configured to carry out a process according to the first aspect of the present invention. All advantages, which have been described in detail in relation to a process according to the present invention according to the first aspect of the present invention, may thus also be provided by the care system according to the present invention according to the second aspect of the present invention, which care system is configured to carry out such a process according to the first aspect of the present invention.

Further, provisions may be made in the care system according to the present invention for the first mobile terminal and/or the second mobile terminal to be configured as one of the following devices:
smartphone;
smartwatch;
tablet (electronic tablet);
pager;
special terminal.

This list is especially not exhaustive, so that other devices may also be used to provide a first mobile terminal and a second mobile terminal, if technically useful and possible. A special terminal may especially be configured as a mobile terminal which is specially constructed for use in a care system according to the present invention.

Further actions improving the present invention appear from the following description of exemplary embodiments of the present invention, which are shown in the figures. All the features and/or advantages appearing from the claims, the description and the drawings, including structural details and arrangements in space, may be essential to the present invention both in themselves and in the different combinations. Components having the same function and mode of operation are provided with the same reference numbers in the drawings. The various features of novelty which characterize the invention are pointed out with particularity in the claims annexed to and forming a part of this disclosure. For a better understanding of the invention, its operating advantages and specific objects attained by its uses, reference is made to the accompanying drawings and descriptive matter in which preferred embodiments of the invention are illustrated.

### BRIEF DESCRIPTION OF THE DRAWINGS

In the drawings:

FIG. 1 is a schematic view showing the care system according to the present invention in case of carrying out step a) of a process according to the present invention;

FIG. 2 is a schematic view showing the care system according to the present invention in case of carrying out step b) of a process according to the present invention;

FIG. 3 is a schematic view showing the care system according to the present invention in case of carrying out step c) of a process according to the present invention;

FIG. 4 is a schematic view showing the care system according to the present invention in case of carrying out step d) of a process according to the present invention; and

FIG. 5 is a schematic view showing the care system according to the present invention in case of carrying out step e) of a process according to the present invention.

### DESCRIPTION OF PREFERRED EMBODIMENTS

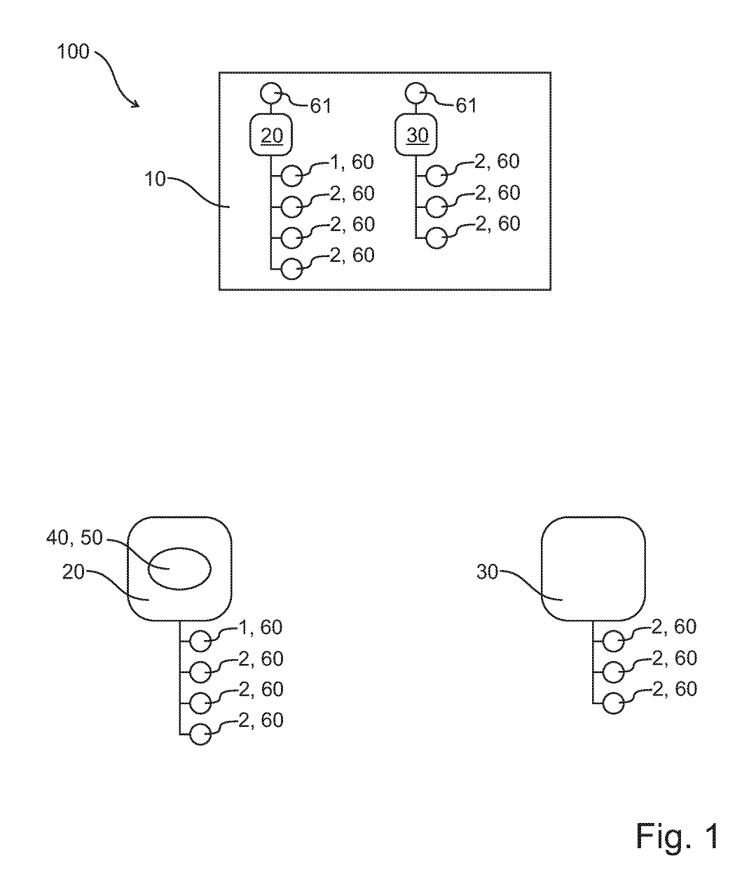

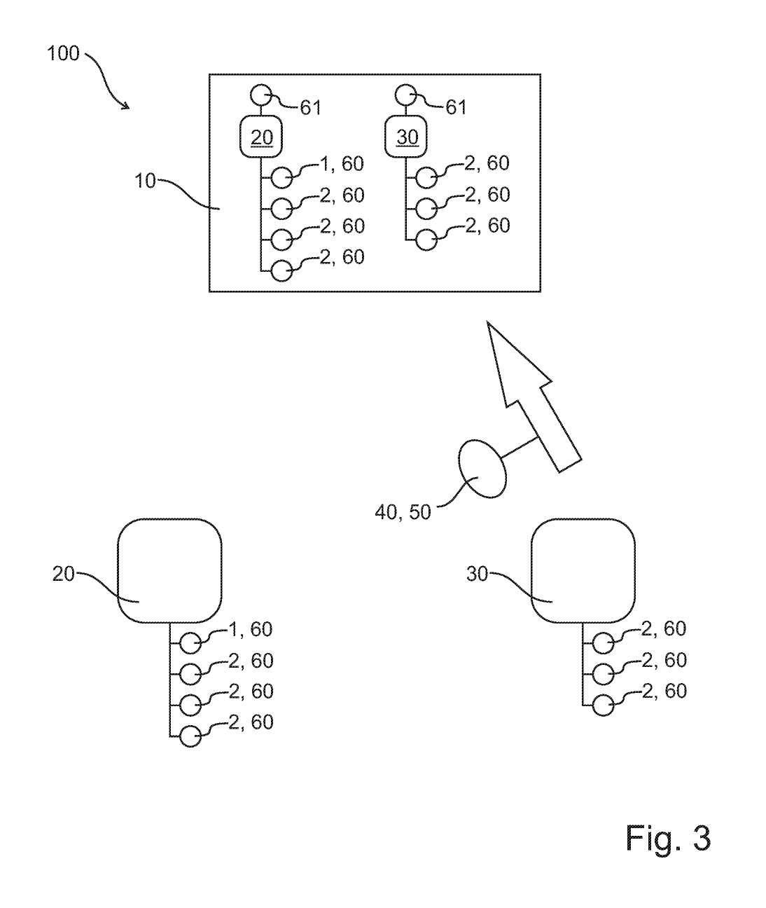

Referring to the drawings, a care system 100 according to the present invention in case of carrying out a process according to the present invention, especially of steps a) through e), is shown in FIGS. 1 through 5. Therefore, the care system 100 is described below and, following this, the individual steps a) through e) of a process according to the present invention are explained in more detail.

A care system 100 according to the present invention especially has a central computer 10 as well as at least one first mobile terminal 20 and a second mobile terminal 30.

The computer 10 comprises one or more processors, associated memory and a device to device communication interface (e.g., a network communication interface as described herein). The mobile terminals 20, 30 may especially be configured, for example, as a smartphone, smartwatch, tablet (electronic tablet), pager or else also as a terminal specially developed for the care system 100 according to the present invention. The mobile terminals 20, 30 comprise a user input/output interface (e.g., one or more of a display, microphone, speaker, keypad, etc.), one or more processors, associated memory, and a device to device interface (e.g., a transmitter/receiver facility). The care system 100 may also have additional mobile terminals. Patients 1, 2 with the corresponding patient data 60 are each assigned to the individual mobile terminals 20, 30. This is shown in FIGS. 1 through 5 both stored in the central computer 10 and also for the individual mobile terminals 20, 30 each. Further, the corresponding user data of a user of the respective mobile terminal 20, 30 are assigned to each of the mobile terminals 20, 30 in the central computer 10. Such user data 61 may be, for example, especially a responsibility of the individual user, training, a work load, a work area or the like. A change 50 of an assignment of the individual patients 1, 2 to the individual mobile terminals 20, 30 can now be carried out by the process according to the present invention described below.

FIG. 1 shows a first step a) of a process according to the present invention. A transfer request 40 is generated by the first mobile terminal 20 or in the first mobile terminal 20. This transfer request 40 pertains to a change 50 of an assignment of at least one assigned patient 1. The transfer request 40 may also contain, for example, additional information, for example, which second mobile terminal 30 has been selected as the recipient of the change 50. The transfer request 40 may be generated, for example, by an input of the user of the first mobile terminal 20. In addition, the transfer request 40, which pertains to a transfer of an assigned patient 1 from the first mobile terminal 20 to the second mobile terminal 30, may be configured as a takeover request, which pertains to a reverse change 50 of an assigned patient 1 from the second mobile terminal 30 (not shown).

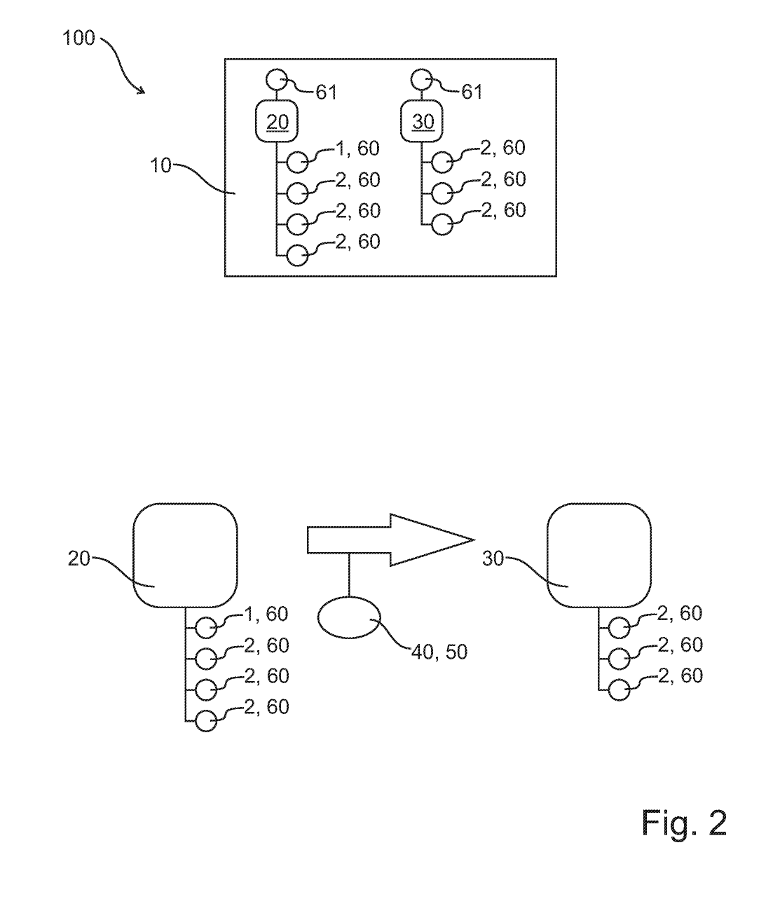

FIG. 2 shows the next step b) of a process according to the present invention, in which the transfer request 40 is especially transmitted from the first mobile terminal 20 to the second mobile terminal 30. This transmission may be carried out, for example, via NFC, WLAN or via a display or reading of a one-dimensional barcode or of a QR code. In particular, the mobile terminals 20, 30 and also the central computer 10 have the necessary devices for this in order to be able to carry out such transmissions. In particular, after carrying out this step b), information is also present in the second mobile terminal 30 to the effect that a change 50 of an assignment of an assigned patient 1 is desired.

This is transmitted to the central computer 10 in the next step c). This can, as shown, be carried out by the second mobile terminal 30. As an alternative and not shown, this transmission may also be carried out from the first mobile terminal 20 or even from both mobile terminals 20, 30. In this way, the central computer 10 can especially be informed about the presence of a transfer request 40 for a change 50 of an assignment of the at least one assigned patient 1.

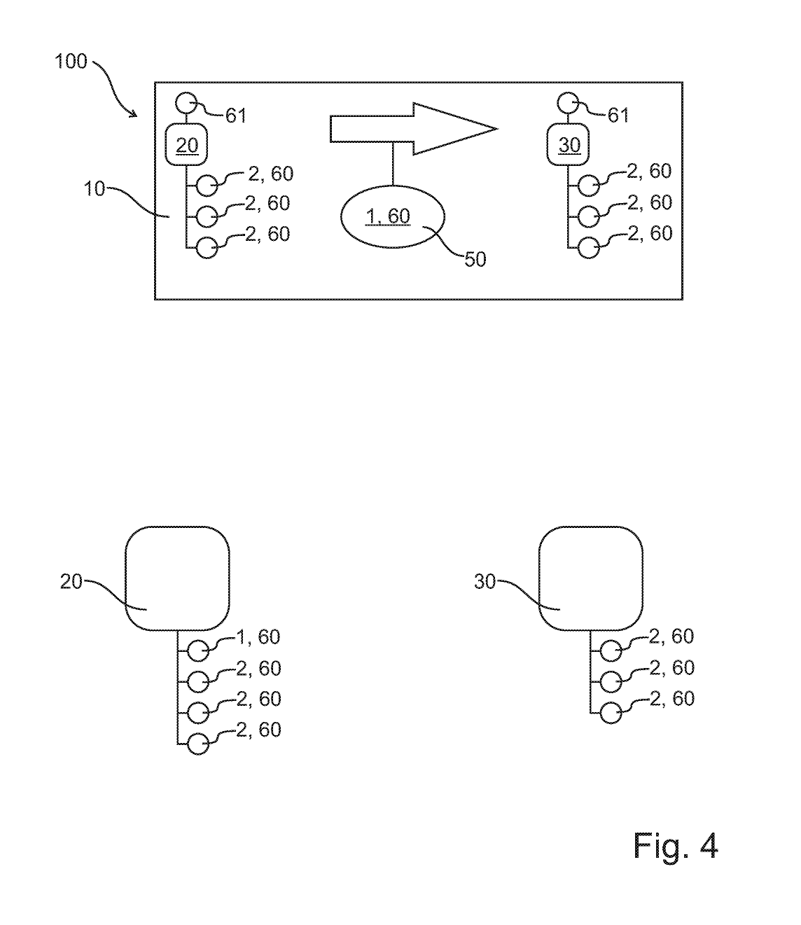

FIG. 4 shows step d) of a process according to the present invention, in which the actual change 50 of the assignment of the at least one assigned patient 1 is carried out. This is especially carried out in the central computer 10, which may also document this change 50 simultaneously or downstream. The central computer 10 may also check an admissibility of the requested change 50, for example, with regard to the patient data 60, especially preferably in relation to the user data 61 of the user of the mobile terminals 20, 30. It can be ensured in this way that a change 50 of the assignment of the at least one assigned patient 1 is not carried out at a user of a mobile terminal 20, 30 who is not trained and/or not authorized for this assignment. A verification or identification of the individual users of the mobile terminals 20, 30 may also be carried out during this checking.

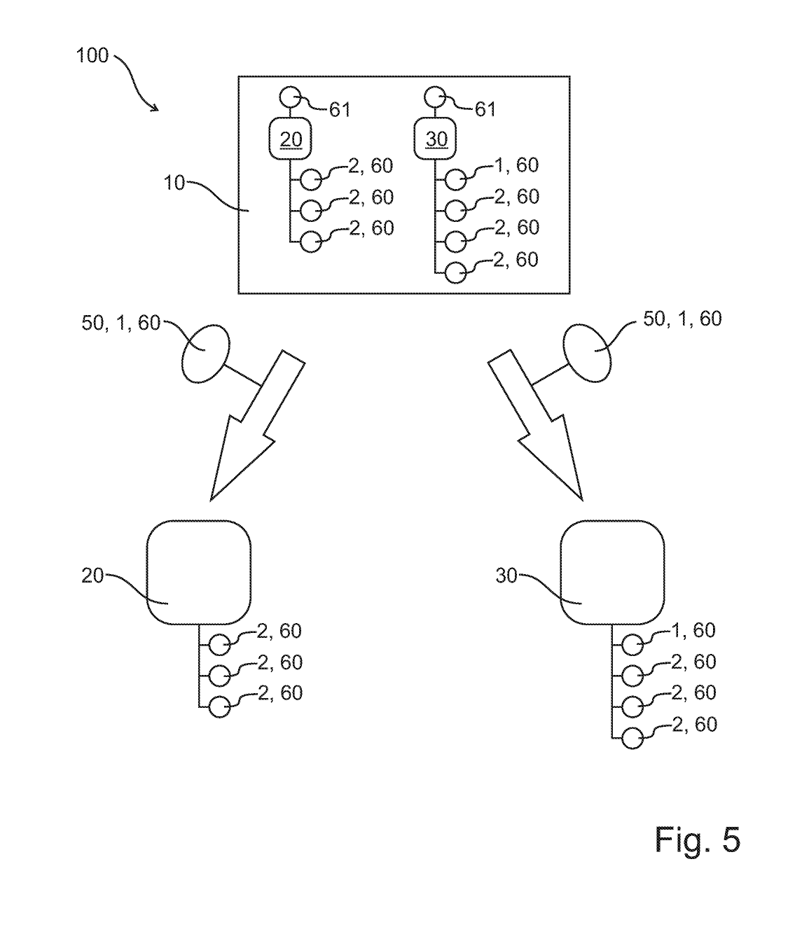

In the last step e), shown in FIG. 5, the change 50 carried out is now transmitted from the central computer 10 again back to the two mobile terminals 20, 30. This transmission may especially also contain patient data 60 of the at least one reassigned patient 1. In addition to the transmission of the change 50 shown, of the at least one reassigned patient 1 and of the patient data 60 from the central computer 10 to the individual mobile terminals 20,30, a direct transmission of, for example, patient data 60 from the first mobile terminal 20 to the second mobile terminal 30 is also conceivable (not shown). As shown, the at least one assigned patient 1 can now be assigned from his assignment to the first mobile terminal 20, see FIG. 1, to the second mobile terminal 30. At the latest now, but preferably already before step c) and/or before step d), a consent to the request change 50 of the assignment can be checked by the second mobile terminal 30. This can be carried out, for example, by an input at a prompt by the user of the second mobile terminal 30. After completion of step e), the change 50 of the assignment of the at least one assigned patient 1 is completed.

Furthermore, the performed change 50 of the at least one assigned patient 1 can be carried out in a manner limited in time and/or in place. In other words, the change 50 of the assignment can be changed back after a defined period of time has passed and/or after, for example, an absence of the first mobile terminal 20 in a defined area has ended. Carrying out a process anew according to the present invention can be avoided in this way. Not only an assigned patient 1, but also a change 50 of a plurality of assigned patients 1 may also be requested in a single transfer request 40. Generation of a respective transfer request 40 for each of these changes 50 can likewise be avoided in this manner. In addition, the transmissions, which have been described in relation to step b) and/or c) and/or e), can especially preferably be carried out in an encrypted manner. Each of the computer and the mobile terminal are configured to following an encryption scheme and possess one or more encryption key to encrypt and decrypt the communication messages. An increase in safety during the carrying out of a process according to the present invention or during the operation of the care system 100 according to the present invention can be increased in this manner.

While specific embodiments of the invention have been shown and described in detail to illustrate the application of the principles of the invention, it will be understood that the invention may be embodied otherwise without departing from such principles.

What is claimed is:

1. A process for carrying out a change of an assignment of patients in a care system on mobile terminals, the process comprising the steps of:
providing the care system so as to comprise: at least one central computer for monitoring and documenting the assignment of the patients; at least one first mobile terminal and a second mobile terminal, wherein patients can each be assigned by the central computer to the mobile terminals;
generating a transfer request, by the first mobile terminal, for a change of assignment of at least one assigned patient;
transmitting the transfer request from the first mobile terminal to the second mobile terminal;
transmitting the transfer request to the central computer;
carrying out, by the central computer, the change of the assignment of the at least one assigned patient; and
transmitting the change of the assignment of the at least one assigned patient to the first mobile terminal and to the second mobile terminal.

2. A process in accordance with claim 1, wherein the steps of transmitting the transfer request from the first mobile terminal to the second mobile terminal, transmitting the transfer request to the central computer and transmitting the change of the assignment is effected with at least one of the following transmission modes: NFC; one-dimensional barcode; QR code; WLAN; wireless device; and mobile cell phone.

3. A process in accordance with claim 1,wherein the step of carrying out, by the central computer, the change of the assignment comprises checking, by the central computer, an admissibility of the requested change of the assignment of the at least one assigned patient.

4. A process in accordance with claim 3, wherein the checking carried out by the central computer pertains to at least one of:
locating the first mobile terminal;
locating the second mobile terminal;
checking patient data of the at least one assigned patient;
checking user data of a user of the first mobile terminal; and
checking user data of a user of the second mobile terminal.

5. A process in accordance with claim 1, wherein the step of carrying out, by the central computer, the change of the assignment comprises a documentation of the change.

6. A process in accordance with claim 1, wherein the step of generating a transfer request comprises a request to change an assignment of two or more assigned patients.

7. A process in accordance with claim 1, wherein the step of transmitting the transfer request to the central computer is carried out by the first mobile terminal and/or by the second mobile terminal.

8. A process in accordance with claim 1, wherein a consent for the requested change of the assignment of the at least one assigned patient is checked by the second mobile terminal before the step of transmitting the transfer request to the central computer or before carrying out, by the central computer, the change of the assignment.

9. A process in accordance with claim 1, wherein the step of carrying out, by the central computer, the change of the assignment of the at least one assigned patient is carried out in a limited and/or in a limited place.

10. A process in accordance with claim 1, wherein patient data of the at least one reassigned patient are transmitted from the central computer to the second mobile terminal.

11. A process in accordance with claim 1, wherein patient data of the at least one reassigned patient are transmitted from the first mobile terminal to the second mobile terminal.

12. A process in accordance with claim 1, wherein at least a portion of transmissions associated with the steps of transmitting the transfer request from the first mobile terminal to the second mobile terminal, transmitting the transfer request to the central computer and transmitting the change of the assignment are encrypted.

13. A process in accordance with claim 1, wherein the assignment of the at least one assigned patient is changed entirely or partially during the change of the assignment of the at least one assigned patient.

14. A process in accordance with claim 1, wherein:
the assignment of the at least one assigned patient is changed during the change of the assignment of the at least one assigned patient from an assignment to the first mobile terminal to an assignment to the second mobile terminal; and/or
the assignment of the at least one assigned patient is changed during the change of the assignment of the at least one assigned patient from an assignment to the second mobile terminal to an assignment to the first mobile terminal.

15. A care system for monitoring patients, the care system comprising:
a plurality of mobile terminals comprising a first mobile terminal and a second mobile terminal, each of the mobile devices comprising a transmitter and a receiver; and
at least one central computer for monitoring and documenting an assignment of the patients being monitored, the at least one central computer comprising a transmitter and a receiver and being configured to assign each of the patients being monitored to the mobile terminals, wherein:
the mobile terminals are configured for the first mobile terminal to generate a transfer request for a change of assignment of at least one assigned patient and transmit the transfer request from the first mobile terminal to the second mobile terminal;
the mobile terminals are configured for one of the first mobile terminal and the second mobile terminal to transmit the transfer request to the central computer;
the central computer is configured to change an assignment of the at least one assigned patient; and
the central computer is configured to transmit the change of the assignment of the at least one assigned patient to the first mobile terminal and to the second mobile terminal.

16. A care system in accordance with claim 15, wherein at least one of the plurality of mobile terminals is configured as one of the following devices: a smartphone; a smartwatch; an electronic tablet; a pager; and a terminal.

17. A care system in accordance with claim 15, wherein the central computer changes the assignment based on determining an admissibility of the requested change of the assignment of the at least one assigned patient.

18. A care system in accordance with claim 15, wherein the checking carried by the central computer pertains to at least one of:
locating the first mobile terminal;
locating the second mobile terminal;
checking patient data of the at least one assigned patient;
checking user data of a user of the first mobile terminal; and
checking user data of a user of the second mobile terminal.

19. A care system in accordance with claim 15, wherein:
carrying out, by the central computer, the change of the assignment comprises a documentation of the change;
consent for the requested change of the assignment of the at least one assigned patient is checked by the second mobile terminal before the step of transmitting the transfer request to the central computer or before carrying out, by the central computer, the change of the assignment; and
transmissions associated with the steps of transmitting the transfer request from the first mobile terminal to the second mobile terminal, transmitting the transfer request to the central computer and transmitting the change of the assignment are encrypted.

20. A care system in accordance with claim 15, wherein:
transmitting the transfer request to the central computer is carried out by the first mobile terminal and/or by the second mobile terminal
generating a transfer request comprises a change of an assignment of two or more assigned patients; and
patient data of the at least one reassigned patient are transmitted from the central computer to the second mobile terminal and/or patient data of the at least one reassigned patient are transmitted from the first mobile terminal to the second mobile terminal and the assignment of the at least one assigned patient is changed during the change of the assignment of the at least one assigned patient from an assignment to the first mobile terminal to an assignment to the second mobile terminal and/or the assignment of the at least one assigned patient is changed during the change of the assignment of the at least one assigned patient from an assignment to the second mobile terminal to an assignment to the first mobile terminal.
